SEQUENCE LISTING (2 sequences)

SEQ ID NO: 1 - protein, 738 aa, ADENO-ASSOCIATED VIRUS 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - DNA, 27 bp, ADENO-ASSOCIATED VIRUS 8
cctgaggggacggcgatgagtcttccg

(19) United States
(12) Patent Application Publication
(10) Pub. No.: US 2022/0348957 A1
(43) Pub. Date: Nov. 3, 2022

(54) ADENO-ASSOCIATED VIRUSES AND THEIR USES FOR INNER EAR THERAPY

(71) Applicants: The United States of America as represented by the Secretary, Department of health and Human Svcs,, Rockville, MD (US); The Trustees of the University of Pennsylvania, Philadelphia, PA (US)

(72) Inventors: Wade W. Chien, Bethesda, MD (US); Jean Bennett, Philadelphia, PA (US)

(73) Assignees: The United States of America as represented by the Secretary, Department of health and Human Svcs., Rockville, MD (US); The Trustees of the University of Pennsylvania, Philadelphia, PA (US)

(21) Appl. No.: 17/435,365

(22) PCT Filed: Jan. 22, 2021
(86) PCT No.: PCT/US21/14553

§ 371 Date: Aug. 31, 2021

Related U.S. Application Data
(60) Provisional application No. 62/965,506, filed on Jan. 24, 2020.

(51) Int. Cl. C12N 15/86; A61P 27/16

(52) U.S. Cl. CPC C12N 15/86; A61P 27/16; C12N 2750/14122; C12N 2750/14143; A61K 48/00

(57) ABSTRACT

Provided herein are adeno-associated viruses and methods for using same to treat or prevent disorders that affect the inner ear of a subject.

### CROSS REFERENCE TO RELATED APPLICATION

This application claims priority to U.S. Provisional Application No. 62/965,506 filed on Jan. 24, 2020, which is incorporated by reference herein in its entirety.

### BACKGROUND

Hearing loss is one of the most common disabilities affecting the world's population today. According to the National Health and Nutritional Examination Survey, nearly two thirds of U.S. adults aged 70 years and older are affected by hearing loss. Hearing loss is often associated with cochlear damage. Inner ear gene therapy is a promising therapeutic modality which can potentially prevent and reverse cochlear damage. Although adeno-associated viral vector (AAV)-mediated inner ear gene therapy has been applied to animal models of hereditary hearing loss to improve auditory function, infection rates in some cochlear cell types are low. In order for inner ear gene therapy to effectively treat hearing loss, a viral vector with higher infection efficiency is required.

### BRIEF SUMMARY OF THE INVENTION

The present disclosure is directed to compositions and methods for treating or preventing diseases or disorders that affect the inner ear of a subject. It has been discovered herein that a recombinant AAV comprising a modified AAV capsid protein can infect the cochlear lateral wall to effectively deliver genetic material into the ear of a subject. In some embodiments, the compositions and methods provided herein can be used to treat or prevent hearing loss and/or dizziness in a subject.

In some embodiments, the present disclosure provides a recombinant adeno-associated virus (AAV) virion comprising: a) a modified AAV capsid protein, wherein the modified AAV capsid protein comprises a mutation relative to a corresponding parental AAV capsid protein, wherein the mutation in the modified AAV capsid protein is in amino acids 587-595 of AAV8-VP1; and b) a heterologous nucleic acid that produces an expression product, wherein the expression product reduces hearing loss or dizziness.

In some embodiments, the expression product is a nucleic acid that decreases expression of a gene associated with hearing loss and/or dizziness, wherein the gene associated with hearing loss and/or dizziness is selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C.

In some embodiments, the expression product is a polypeptide that reduces hearing loss and/or dizziness, wherein the polypeptide is selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPL5, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A.

In some embodiments, the recombinant AAV virion is selected from the group consisting of AAV2, AAV5, AAV8 and AAV9. In some embodiments, the recombinant AAV virion is an AA8 virion comprising a modified AAV8-VP1 capsid protein, for example, a AAV8BP2 virion.

In some embodiments, the expression product is a nucleic acid that decreases expression of a gene associated with hearing loss and is an interfering RNA. In some embodiments, the interfering RNA is an antisense molecule, a short interfering RNA or an miRNA.

In some embodiments, the AAV virions produce an expression product that reduces age-related hearing loss, hereditary hearing loss, noise-induced hearing loss, hearing loss as the result of administration of ototoxic drugs, disease-associated hearing loss or hearing loss resulting from trauma.

In another embodiment, the present disclosure provides a method for treating or preventing cochlear damage in a subject comprising administering to the subject having cochlear damage or at risk of developing inner ear hair cell damage, an effective amount of any recombinant AAV virion described herein.

In some embodiments, the subject has or is at risk of developing age-related hearing loss, hereditary hearing loss, noise-induced hearing loss, hearing loss as the result of administration of ototoxic drugs, disease-associated hearing loss and hearing loss resulting from trauma.

In some embodiments, the recombinant AAV virion infects the cochlear lateral wall of the subject. In some embodiments, the recombinant AAV virion infects the stria vascularis in the cochlear lateral wall. In some embodiments, the recombinant AAV virion infects the marginal cells of the stria vascularis.

In some embodiments, the recombinant AAV virion increases inner ear hair cell regeneration, for example, cochlear hair cell regeneration.

In another embodiment, the present disclosure also provides a method for treating or preventing hearing loss and/or dizziness in a subject, comprising administering to the subject having hearing loss and/or dizziness or at risk of developing hearing loss and/or dizziness, an effective amount of any recombinant AAV virion described herein.

In some embodiments, the subject having hearing loss or at risk of developing hearing loss is a subject that has or is at risk of developing age-related hearing loss, hereditary hearing loss, noise-induced hearing loss, hearing loss as the result of administration of ototoxic drugs, disease-associated hearing loss and hearing loss resulting from trauma.

In some embodiments, the recombinant AAV virion infects the cochlear lateral wall of the subject of the subject that has or is at risk of developing hearing loss. In some embodiments, the recombinant AAV virion infects the stria vascularis in the cochlear lateral wall of the subject. In some embodiments, the recombinant AAV virion infects the marginal cells in the stria vascularis of the subject.

In any of the methods provided herein, the recombinant AAV virion can be administered to the subject intravenously, intrathecally, intratympanically, via round window administration, via semicircular canal delivery, or via stapedotomy. In some embodiments, the recombinant AAV virion is administered via canalostomy into the posterior semicircular canal of the subject.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1a-1e show cryosections of the cochlea after AAV-GFP injections via PSCC. (a) AAV2.7m8 transduced many cell types on the sensory epithelium, but not in the lateral wall. (b) AAV8BP2 transduced cells in the lateral wall (white arrow). (c) Anc80 transduced cells in the spiral prominence (white arrow). (d) AAV2 did not transduce cells in the lateral wall. (e) AAV8 transduced cells in the lateral wall.

FIG. 2 is a graph providing quantifications of marginal cell infection efficiency for AAV2.7m8, AAV2, Anc80L65, AAV8, and AAV8BP2. AAV8BP2 transduced the marginal cells most efficiently and showed statistically significant differences compared to AAV2, AAV2.7m8, and Anc80L65 (p<0.05).

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

In accordance with the present disclosure it has been discovered that a recombinant AAV comprising a modified AAV capsid protein can infect the cochlear lateral wall to effectively deliver genetic material into the cochlear lateral wall of a subject. Provided herein are compositions and methods for treating or preventing cochlear damage. In some embodiments, the compositions and methods provided herein can be used to treat or prevent diseases or disorders that affect the inner ear of the subject.

In some embodiments, the recombinant AAV virion comprises a) a modified AAV capsid protein, wherein the modified AAV capsid protein comprises a mutation relative to a corresponding parental AAV capsid protein, wherein the mutation in the modified AAV capsid protein is in the position corresponding to amino acids 587-595 of AAV8-VP1; and b) a heterologous nucleic acid that produces an expression product, wherein the expression product reduces hearing loss or dizziness.

The modified AAV capsids can comprise one or more mutations in amino acids 587-595 of AAV8-VP1 or the corresponding positions of the capsid subunit of another AAV serotype. One of skill in the art could readily align the amino acid sequence of AAV8-VP1 with the amino acid sequence of a VP1 amino acid sequence of another AAV serotype to identify amino acids corresponding to amino acids 587-595 of AAV8-VP1 in a VP1 from another AAV serotype, for example, in a VP1 from AAV1, AAV2, AAV3, AAV4, AAV5, AAV6, AAV7, AAV9 or AAV10. The amino acid sequence for AAV8 VP1 capsid protein is provided herein as SEQ ID NO: 1:

(SEQ ID NO: 1)

MAADGYLPDW LEDNLSEGIR EWWALKPGAP KPKANQQKQD
DGRGLVLPGY KYLGPFNGLD KGEPVNAADA AALEHDKAYD
QQLQAGDNPY LRYNHADAEF QERLQEDTSF GGNLGRAVFQ
AKKRVLEPLG LVEEGAKTAP GKKRPVEPSP QRSPDSSTGI
GKKGQQPARK RLNFGQTGDS ESVPDPQPLG EPPAAPSGVG
PNTMAAGGGA PMADNNEGAD GVGSSSGNWH CDSTWLGDRV
ITTSTRTWAL PTYNNHLYKQ ISNGTSGGAT NDNTYFGYST
PWGYFDFNRF HCHFSPRDWQ RLINNNWGFR PKRLSFKLFN
IQVKEVTQNE GTKTIANNLT STIQVFTDSE YQLPYVLGSA
HQGCLPPFPA DVFMIPQYGY LTLNNGSQAV GRSSFYCLEY
FPSQMLRTGN NFQFTYTFED VPFHSSYAHS QSLDRLMNPL
IDQYLYYLSR TQTTGGTANT QTLGFSQGGP NTMANQAKNW
LPGPCYRQQR VSTTTGQNNN SNFAWTAGTK YHLNGRNSLA
NPGIAMATHK DDEERFFPSN GILIFGKQNA ARDNADYSDV
MLTSEEEIKT TNPVATEEYG IVADNLQQQN TAPQIGTVNS
QGALPGMVWQ NRDVYLQGPI WAKIPHTDGN FHPSPLMGGF
GLKHPPPQIL IKNTPVPADP PTTFNQSKLN SFITQYSTGQ
VSVEIEWELQ KENSKRWNPE IQYTSNYYKS TSVDFAVNTE
GVYSEPRPIG TRYLTRNL

In some embodiments, one or more mutations can be made between amino acids corresponding to 587-595 of SEQ ID NO: 1, inclusive of the amino acid corresponding to position 587 of SEQ ID NO: 1 and the amino acid corresponding to amino acid 595 of SEQ ID NO: 1, or of an amino acid sequence having at least 75%, 80%, 90%, 95% or 99% identity to the amino acid sequence of AAV8-VP1.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

In some embodiments, the recombinant AAV virion is an AAV8BP2 virion. See, for example, Cronin et al. (Efficient transduction and optogenetic stimulation of retinal bipolar cells by a synthetic adeno-associated virus capsid and promoter, EMBO Mol. Med. 6(9): 1175-1190 (2014)) and U.S. Patent Application Publication No. 20150376240, hereby incorporated by reference in their entireties. In AAV8BP2 virions, the VP1 capsid sequence comprises a nucleotide sequence comprising SEQ ID NO: 2 (CCT GAG GGG ACG GCG ATG AGT CTT CCG). SEQ ID NO: 2 encodes amino acids 585-594 of AAV8VP1 in an AAV8BP2 virion. In some embodiments, the recombinant AAV virion provides for increased infectivity of the cochlear lateral wall of the ear of a subject, for example, in marginal cells of the stria vascularis, as compared to the infectivity of the cochlear lateral wall by a recombinant AAV virion comprising the corresponding parental AAV capsid protein that does not have one or more mutations in the VP1 capsid protein. In some embodiments, the recombinant AAV virion, for example, AAV8BP2, provides for increased infectivity of the cochlear lateral wall, for example in a marginal cell of the stria vascularis, as compared to the infectivity of the marginal cell by a recombinant AAV2.7m8 virion or a recombinant AAV Anc80L65 virion. Increased infectivity of the cochlear lateral wall after administration of a recombinant AAV virion described herein, for example, AAV8BP2, can be at least about a 5%, 10%, 20%, 30%, 40%, 50%, 60%, 70%, 80%, 90%, 100% increase or greater as compared to a control. The increase can also be at least a 2-fold, 5-fold, 10-fold, 20-fold, 30-fold, 40-fold, 50-fold increase or greater.

As used herein, a recombinant AAV virion is a viral particle comprising at least one AAV capsid protein and an encapsulated recombinant AAV vector. As used herein, a “recombinant AAV vector” refers to an AAV vector comprising a nucleic acid sequence that is not normally present in AAV (i.e., a polynucleotide heterologous to AAV), for example, a nucleic acid sequence of interest for genetic transformation of a cell. In general, the heterologous nucleic acid is flanked by at least one, and generally by two, AAV inverted terminal repeat sequences (ITRs). The term recombinant AAV vector encompasses both rAAV vector particles and recombinant AAV vector plasmids. A recombinant AAV vector may either be single-stranded (ssAAV) or self-complementary (scAAV).

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC 002077 (AAV-1), AF063497 (AAV-1), NC 001401 (AAV-2), AF043303 (AAV-2), NC 001729 (AAV-3), NC 001829 (AAV-4), U89790 (AAV-4), NC 006152 (AAV-5), AF513851 (AAV-7), AF513852 (AAV-8), and NC 006261 (AAV-8); the disclosures of which are incorporated by reference herein for teaching AAV nucleic acid and amino acid sequences.

An “AAV virus,” “AAV virion,” “AAV viral particle,” or “recombinant AAV vector particle” refers to a viral particle composed of at least one AAV capsid protein and an encapsidated polynucleotide recombinant AAV vector. If the particle comprises a heterologous nucleic acid sequence (i.e., a nucleic acid sequence other than a wild-type AAV genome such as a transgene to be delivered to a mammalian cell), it can be referred to as a recombinant AAV vector. Thus, production of recombinant AAV particles or virions necessarily includes production of a recombinant AAV vector, as such a vector is contained within a recombinant AAV particle. Methods for producing AAV vectors and virions are known in the art. See, for example, Shin et al. “Recombinant Adeno-Associated Viral Vector Production and Purification,” Methods Mol. Biol. 798: 267-284 (2012)). Any of the AAV virions described herein can be used to infect one or more cell types in lateral wall of the cochlea, for example in the stria vascularis (for example marginal cells, intermediate cells or basal cells of the stria vascularis). Optionally, any of the virions described herein can infect one or more types of inner ear hair cells, including, but not limited to cochlear cells, vestibular cells, inner hairs cell of the cochlea, outer hair cells of the cochlea, glia-like supporting cells of the cochlea (for example, Hensen's cells, Deiters' cells, inner and outer pillar cells, Claudius cells and inner phalangeal cells). In some embodiments, a AAV virion that infects the stria vascularis, for example, marginal cells of the stria vascularis, is administered the subject in combination with an AAV virion, for example, an AAV2.7m8 virion, that infects one or more types of inner ear hair cells, including, but not limited to cochlear cells, vestibular cells, inner hairs cell of the cochlea, outer hair cells of the cochlea, glia-like supporting cells of the cochlea (for example, Hensen's cells, Deiters' cells, inner and outer pillar cells, Claudius cells and inner phalangeal cells).

As used throughout, a “corresponding parental AAV capsid protein” refers to an AAV capsid protein of the same AAV serotype, without the peptide insertion. As used herein, when describing recombinant AAV vectors or virions, the phrase “heterologous” refers to a nucleic acid sequence not naturally found in wild-type AAV. For example, a heterologous nucleic acid that produces an expression product is a nucleic acid not normally found in a wild-type AAV. In embodiments where the heterologous nucleic acid encodes a polypeptide, the encoded polypeptide is a heterologous polypeptide not normally encoded or expressed by a naturally-occurring, wild-type AAV.

As used throughout, an “expression product” is a nucleic acid or a polypeptide that is expressed or produced in a cell, for example, an inner ear hair cell, after infection by an AAV virion. The expression product can be expressed by infecting cells in vitro, in vivo or ex vivo. As used in this specification and the appended claims, the singular forms “a,” “an,” and “the” include plural reference unless the context clearly dictates otherwise. Therefore, the terms “a virion” or “a cell” also refer to more than one virion or cell, for example, populations of virions or cells.

Expression products include, but are not limited to, a polypeptide, an aptamer, an antisense molecule, an interfering RNA or an mRNA. In some embodiments, the expression product is an interfering RNA selected from the group consisting of an short interfering RNA (siRNA), a short hairpin (shRNA) and an miRNA.

As used throughout, the term “nucleic acid” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In some embodiments, a nucleic acid encoding an expression product of interest is operably linked to a constitutive promoter. In other embodiments, a nucleic acid encoding an expression product of interest is operably linked to an inducible promoter. In some instances, a nucleic acid encoding an expression product of interest is operably linked to a tissue-specific or cell type-specific regulatory element. For example, in some instances, a nucleic acid encoding an expression product of interest is operably linked to an inner ear hair cell-specific regulatory element, e.g., a regulatory element that confers selective expression of the operably linked nucleic acid in an inner ear hair cell. See, for example, Boeda and Petit “A specific promoter of the sensory cells of the inner ear defined by transgenesis” Hum Mol. Genet. 19(15): 1581-9 (2001), for expression of a gene product under the control of the MYO7A promoter in inner ear hair cells. As used herein, specific expression does not mean that the expression product is expressed only in a specific tissue(s) or cell type(s), but refers to expression substantially limited to specific tissue(s) or cell types(s). Any heterologous nucleic acid that produces an expression product can further comprise a nucleic acid encoding a detectable polypeptide, for example, a fluorescent polypeptide (GFP, RFP, etc.) or an active fragment thereof.

Upon infection of the cochlear lateral wall, for example, in marginal cells of the stria vascularis, or any other cell type described herein, with any of the AAV virions described herein, the expression product produced in the cochlear lateral wall, for example, in marginal cells of the stria vascularis, or any other cell type described herein reduces hearing loss and/or dizziness in the subject. In some embodiments, the expression product is a nucleic acid, for example, an antisense molecule or an interfering RNA, that decreases expression of a gene associated with hearing loss and/or dizziness in a subject.

In some embodiments, a nucleic acid, for example, an antisense molecule or an interfering RNA, decreases expression of one or more genes selected from the group consisting of DIAPH1, KCNQ4, GJB3, IFNLR1, GJB2, GJB6, MYH1, CEACAM16, GSDME/DFNA5, WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C in an inner ear hair cell of subject. In some embodiments, a decrease in expression is a decrease in transcription of mRNA and/or a decrease in translation of a polypeptide or a fragment thereof translated from an mRNA. The decrease or reduction in expression can be a decrease or reduction of about 5%, 10%, 20%, 30%, 40%, 50%, 60%, 70%, 80%, 90%, 100% or any percentage in between these percentages as compared to a control. By reducing expression of one or more genes selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C, hearing loss can be reduced or improved.

In some embodiments, the expression product is a polypeptide that reduces or improves hearing loss and/or dizziness in a subject. As used throughout, “polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds. Fragments of any of the polypeptides described herein are also encompassed by these terms.

In some embodiments, one or more polypeptides selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPL5, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A or a fragment thereof are expressed in an inner ear hair cell of a subject.

In some embodiments, upon infection of the cochlear lateral wall, for example, in a marginal cell of the stria vascularis, or any other cell type described herein by a recombinant AAV virion described herein, there is at least a 2-fold, at least 5-fold, at least 10-fold, at least 20-fold, at least 30-fold, at least 40-fold, at least 50-fold or more than at least a 50-fold increase in the level of one or more polypeptides in the marginal cell of the subject as compared to control, such that hearing loss and/or dizziness in a subject is reduced.

The expression product can be heterologous to the cell in the subject. As used herein the phrase “heterologous,” as it relates to the expression product in a cell, for example, an inner ear hair cell of the subject, refers to a nucleic acid or a polypeptide not naturally found in a cell of the subject. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

### Methods

Provided herein are methods for delivering a nucleic acid of interest to the inner ear by administering any of the AAV virions described herein. In some embodiments, the AAV virion comprises a nucleic acid of interest. In some embodiments, only the AAV virion is delivered to the subject. In some embodiments, the nucleic acid of interest is delivered to the cochlear lateral wall of the subject, for example to cells in the stria vascularis. In some embodiments, AAV virion or the AAV virion comprising the nucleic acid of interest is delivered to marginal cells in the stria vascularis. In some embodiments, the AAV virion is an AAV8BP2 virion comprising the nucleic acid of interest. In some embodiments, the nucleic acid of interest decreases cochlear damage, inner hair cell damage, hearing loss and/or dizziness. In some embodiments, the nucleic acid of interest encodes a polypeptide that decreases cochlear damage, inner hair cell damage, hearing loss and/or dizziness.

Hearing loss is often caused by cochlear damage, for example, by damage to inner ear hair cells, such as, cochlear hair cells. The mammalian cochlea contains two types of hair cells, inner hair cells (IHCs) and outer hair cells (OHCs), both of which are important for the detection and processing of auditory information. These hair cells are surrounded by supporting cells, a heterogeneous group of cells which are important for cochlear homeostasis. The mature mammalian hair cells are incapable of regeneration. Therefore, once the damage occurs in these cells, the degeneration process is often irreversible.

Provided herein is a method of treating or preventing cochlear damage in a subject comprising administering to the subject having cochlear damage or at risk of developing inner ear hair cell damage, an effective amount of a recombinant AAV virion described herein. In some embodiments, the recombinant virion is a recombinant AAV virion, for example, an AAV8BP2 virion, comprising a nucleic acid that decreases expression of a gene associated with inner ear hair cell damage. In some embodiments, the recombinant AAV virion is a recombinant AAV virion, for example, an AAV8BP2 virion, comprising a nucleic acid encoding a polypeptide that treats or prevents inner ear hair cell damage in a subject. In some embodiments, the subject having cochlear damage or at risk of developing cochlear damage, has hearing loss or is at risk of developing hearing loss. In some embodiments, the subject having cochlear damage or at risk of developing cochlear damage experiences dizziness.

In another embodiment, provided herein is a method of treating or preventing hearing loss and/or dizziness in a subject, comprising administering to the subject having hearing loss or dizziness or at risk of developing hearing loss or dizziness, an effective amount of a recombinant AAV virion described herein. In some embodiments, the recombinant AAV virion is a recombinant AAV8 virion, for example, an AAV8BP2 virion, comprising a nucleic acid that decreases expression of a gene associated with inner ear hair cell damage. In some embodiments, the recombinant virion is a recombinant AAV virion, for example, an AAV8BP2 virion, comprising a nucleic acid encoding a polypeptide that treats or prevents inner ear hair cell damage in a subject.

In some embodiments, the recombinant AAV virion increases inner ear hair cell regeneration, for example, cochlear hair cell regeneration. In some embodiments, the recombinant AAV virion infects the cochlear lateral wall. In some embodiments, the recombinant AAV virion infects the stria vascularis in the cochlea lateral wall. In some embodiments, the recombinant AAV virion infects marginal cells in the stria vascularis in the cochlear lateral wall. In some embodiments, the recombinant AAV virion increases regeneration of inner hair cells, outer hair cells and/or glia-like supporting cells of the cochlea. In some embodiments, the recombinant AAV virion preferentially infects marginal cells in the stria vascularis. In some embodiments, the recombinant AAV virion infection efficiency in marginal cells in the stria vascularis of the subject is at least 5%, 10%, 20%, 30%, 40%, 50%, 60%, 70%, 80%, 90% or at least 100% higher than the recombinant AAV virion infection efficiency in other cochlear cells of the subject. In some embodiments, the level of the expression product produced by the recombinant AAV virion in marginal cells of the stria vascularis in the subject is at least 5%, 10%, 20%, 30%, 40%, 50%, 60%, 70%, 80%, 90% or at least 100% higher in marginal cells as compared to other cochlear cells of the subject.

The methods and compositions provided herein can be used to treat a subject having or at risk of developing any type of hearing loss. Hearing loss can be on the level of conductivity, sensorineural and/or central level. Conductive hearing loss is caused by lesions involving the external or middle ear, resulting in the destruction of the normal pathway of airborne sound amplified by the tympanic membrane and the ossicles to the inner ear fluids. Sensorineural hearing loss is caused by lesions of the cochlea or the auditory division of the eight cranial nerve. Central hearing loss is due to lesions of the central auditory pathways. In some cases, conductive hearing loss occurs in combination with sensorineural hearing loss (mixed hearing loss).

The compositions and methods provided herein can be used to treat subjects having or at risk of developing age-related hearing loss (presbycusis), hereditary hearing loss, noise-induced hearing loss, disease-associated hearing loss, exposure to toxic substances, hearing loss as the result of administration of ototoxic drugs, and hearing loss resulting from trauma, to name a few.

In some embodiments, hereditary hearing loss can be caused by a mutation in one or more genes involved in hearing. Some mutations cause hearing loss that is non-syndromic, meaning that the subject does not have any other symptoms except hearing loss. Other mutations causing hearing loss are syndromic, meaning that the person has other symptoms besides hearing loss (for example, Waardenburg's syndrome, Alport's syndrome and Usher's syndrome). In some embodiments, the hereditary hearing loss is autosomal dominant hearing loss, for example, hearing loss caused by a mutation in the GJB2.

In some embodiments, a nucleic acid encoding a non-mutated polypeptide of a missing or mutated gene associated with hearing loss is delivered to any inner ear cell described herein, for example, the marginal cells of the stria vascularis in the subject, to provide the marginal cells with a working copy of a missing or mutated gene involved in hearing loss. In other embodiments, a nucleic acid that decreases expression of a one or more mutant alleles of a gene involved in hearing loss is delivered to any inner ear cell described herein, for example, the marginal cells in the stria vascularis of the subject.

The compositions and methods provided herein can also be used to treat a subject having or at risk of developing dizziness. In some embodiments, dizziness is associated with a vestibular disorder. Examples of vestibular disorders include, but are not limited to, benign paroxysmal positional vertigo (BPPV), labyrinthitis. vestibular neuritis, Ménière's disease, secondary endolymphatic hydrops, and perilymph fistula. Vestibular disorders also include superior canal dehiscence, acoustic neuroma, ototoxicity, enlarged vestibular aqueduct syndrome, and mal de débarquement.

Any of the methods of treating hearing loss or dizziness provided herein can be combined with other treatments for hearing loss or dizziness, for example, a hearing aid, administration of an effective amount of a corticosteroid, or exercises for treating vertigo, to name a few.

Throughout, treat, treating, and treatment refer to a method of reducing or delaying one or more effects or symptoms of hearing loss (e.g., trouble understanding speech, listening to television or radio at high volume, tinnitus, asking people to repeat themselves) or dizziness (e.g., loss of balance, fainting, double vision, confusion, slurred speech, numbness in arms or legs). The subject can be diagnosed with hearing loss or dizziness. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is at least about a 10% reduction in hearing loss or dizziness in a subject when compared to the subject prior to treatment or when compared to a control subject or control value. Thus, the reduction can be about a 10, 20, 30, 40, 50, 60, 70, 80, 90, 100%, or any amount of reduction in between. A reduction in hearing loss can also be a percentage improvement in hearing of at least about 5, 10, 20, 30, 40, 50, 60, 70, 80, 90, 100%, or any percentage in between these percentages. Methods for testing hearing in a subject are known in the art and include,

As used herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of hearing loss or dizziness or one or more symptoms of hearing loss (e.g., trouble understanding speech, listening to television or radio at high volume, tinnitus, asking people to repeat themselves) or dizziness (e.g., loss of balance, fainting, double vision, confusion slurred speech, numbness in arms or legs) in a subject susceptible to hearing loss or dizziness compared to control subjects susceptible to hearing loss or dizziness that did not receive treatment. The reduction or delay in onset, incidence, severity, or recurrence of hearing loss or dizziness can be about a 10, 20, 30, 40, 50, 60, 70, 80, 90, 100%, or any amount of reduction in between.

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

### Pharmaceutical Compositions

Provided herein is a pharmaceutical composition comprising any of the recombinant AAV virions described herein and a pharmaceutically acceptable carrier, diluent, excipient, or buffer. In some embodiments, the pharmaceutically acceptable carrier, diluent, excipient, or buffer is suitable for use in a subject, for example, a human. The pharmaceutical compositions can be delivered to a subject, so as to allow production of an expression product in an inner ear cell of the subject. Pharmaceutical compositions comprise sufficient genetic material that allows the recipient to produce an effective amount of an expression product that reduces or prevents inner hair cell damage. In some embodiments, the pharmaceutical compositions comprise sufficient genetic material that allows the recipient to produce an effective amount of an expression product that treats or prevents hearing loss and/or dizziness in a subject.

The compositions may be administered alone or in combination with at least one other agent, such as stabilizing compound, which may be administered in any sterile, biocompatible pharmaceutical carrier, including, but not limited to, saline, buffered saline, dextrose, and water. In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

### Delivery Methods

The present disclosure provides a method of delivering an expression product to an inner ear cell in an individual, the method comprising administering to the individual a recombinant AAV virion as described above. The expression product can be a polypeptide, an antisense molecule, an interfering RNA or an aptamer, to name a few.

The term “effective amount,” as used throughout, is defined as any amount necessary to produce a desired physiologic response, for example, reducing or preventing inner ear hair cell damage. Effective amounts and schedules for administering the recombinant AAV virions described herein can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

An effective amount of any of the recombinant AAV virions described herein will vary and can be determined by one of skill in the art through experimentation and/or clinical trials. For example, for in vivo injection, for example, injection directly into the inner ear of a subject, an effective dose can be from about 10⁶ to about 10¹⁵ recombinant rAAV virions, for example, from about 10⁸ to 10¹² recombinant AAV virions. For in vitro infection, an effective amount of recombinant virions to be delivered to cells can be from about 10⁶ to about 10¹⁵ of the recombinant AAV virions. Other effective dosages can be readily established by one of ordinary skill in the art through routine trials establishing dose response curves.

The compositions described herein are administered in a number of ways depending on whether local or systemic treatment is desired. The compositions are administered via any of several routes of administration, intravenously, intrathecally, intratympanically, via round window administration, via semicircular canal delivery, or via stapedotomy. In some embodiments, the compositions are administered canalostomy into the posterior semicircular canal of the subject. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1

Adeno-associated virus (AAV) is a commonly used viral vector for gene therapy studies due to its excellent biosafety profile. It has been used successfully in several inner ear gene therapy studies to improve auditory and vestibular functions in mutant mice. While most studies on AAV inner ear gene delivery have focused on transducing the mechanosensory hair cells and supporting cells, few studies have examined AAV transduction in the cochlear lateral wall.

The stria vascularis in the cochlear lateral wall is responsible for maintaining the endocochlear potential, which is critical for normal cochlear function. Some of the most common types of hereditary hearing loss are caused by variants in genes which are expressed in the cochlear lateral wall (e.g. SLC26A4)7. In this example, the lateral wall transduction patterns of two conventional AAVs (AAV2 and AAV8) and three synthetic AAVs (AAV2.7m8, AAV8BP2, Anc80L65) in the neonatal mouse inner ear were studied.

### Methods

### AAV Vector Construction

The AAV2.7m8-CAG-eGFP, AAV8BP2-CAG-eGFP, and Anc80L65-CAG-eGFP were produced by the Research Vector Core at the Center for Advanced Retinal and Ocular Therapeutics (University of Pennsylvania). The production method for these viruses are described in Ramachandran et al. (Evaluation of Dose and Safety of AAV7m8 and AAV8BP2 in the Non-Human Primate Retina. Hum Gene Ther 28, 154-167 (2017)). The concentration of viral stock solution was 1×10¹³ genome copies (G.C.) per ml for each virus.

### Animal Surgery

P0-P5 neonatal CBA/J mice were used in the study. Three synthetic (AAV2.7m8, AAV8BP2, and Anc80L65) and two conventional (AAV2 and AAV8) AAV-GFPs were delivered through the posterior semicircular canal using a glass micropipette. 1 ml was injected into the left ear of each mouse.

### Auditory Brainstem Response

Auditory brainstem response (ABR) testing was used to evaluate hearing sensitivity. ABRs were recorded using the Tucker Davis Technologies system (RZ6 Multi I/O Processor, Tucker-Davies Technologies, Gainesville, Fla., USA) on CBA/J mice at 1 month of age. Mice were anesthetized using ketamine and dexdomitor, and placed on a heating pad set to 37° C. ABR thresholds were measured at 4, 8, 16, and 32 kHz. Between 512-1024 responses were averaged at each stimulus level.

### Immunohistochemistry

Whole mounts of the cochlea were obtained and dissected into base, middle, and apical turns. The lateral wall from the base and middle was dissected out separately. Antibodies against SLC12a2 and Hoechst/Phalloidin were applied to lateral wall whole mounts. Confocal microcopy using a Zeiss LSM780 was used to take Z-stacks images of the marginal cells which were then quantified based on their marginal cell markers SLC12a2/Phalloidin and GFP expression. Cryosections of the cochlea were collected and sectioned using a Leica CM3050 cryostat.

### Results

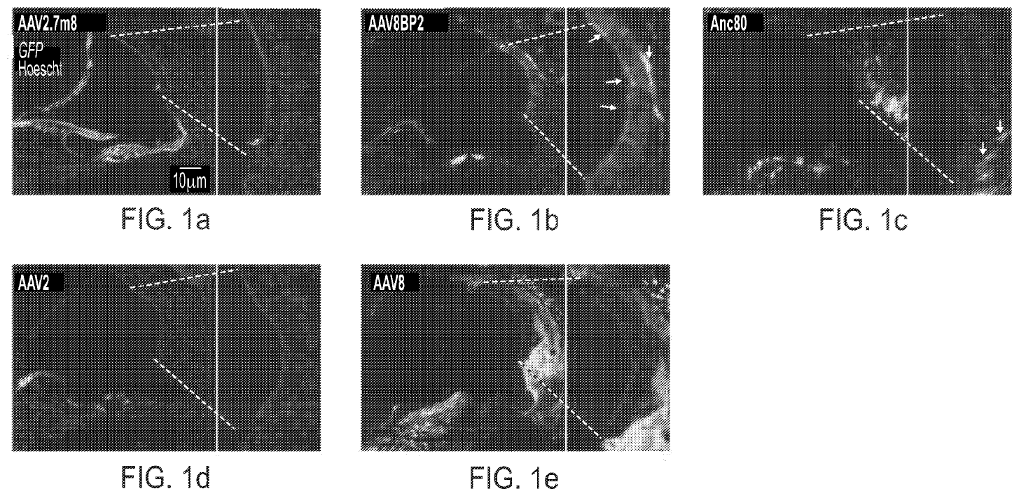

The lateral wall transduction patterns of two conventional AAVs (AAV2 and AAV8) and three synthetic AAVs (AAV2.7m8, AAV8BP2, Anc80L65) in the neonatal mouse inner ear were examined. As shown in FIG. 1a, AAV2.7m8 transduced many cell types on the sensory epithelium, but not in the cochlear lateral wall. FIG. 1b shows that AAV8BP2 transduced cells in the lateral wall (white arrow). Anc80 injections resulted in some infection near the spiral prominence (white arrow in FIG. 1c). FIG. 1d shows that, similar to AAV2.7m8, AAV2 did not transduce cells in the lateral wall. AAV8 injections resulted in some infections in the lateral wall (FIG. 1e).

AAV8 and AAV8BP2 transduced the marginal cells in the stria vascularis. AAV8BP2-GFP injection resulted in high levels of GFP expression in marginal cells (MC). AAV8-GFP injected mice expressed relatively higher levels. In contrast, AAV2-GFP, AAV2.7m8, and Anc80L65-GFP transduced marginal cells at lower levels.

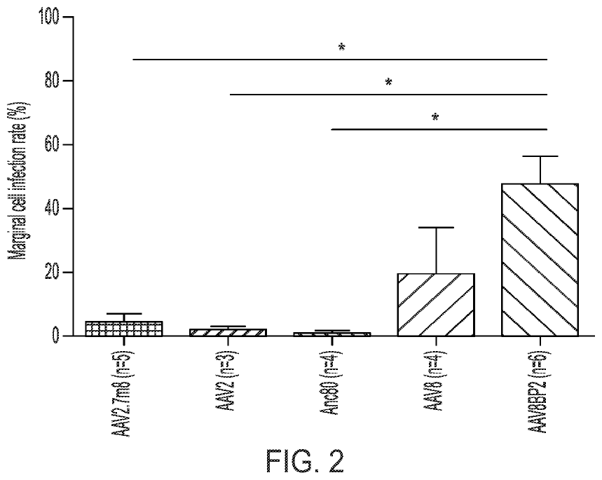

Upon quantification of marginal cell transduction efficiency, it was shown that AAV8BP2 transduced marginal cells of the stria vascularis most effectively (FIG. 2).

In order to utilize gene therapy to maintain or improve cochlear function, one critical element is to have a viral vector which can effectively target the cochlear lateral wall. As shown herein, AAV8BP2 effectively infects the cochlear lateral wall. In addition, it infects the marginal cells of the stria vascularis which is responsible for maintaining endocochlear potential. These results demonstrate that AAV8BP2 is a powerful viral vector that can greatly expand the applications for inner ear gene therapy.

### Example 2

### Methods

### AAV Vector Construction

The AAV8BP2-CAG-eGFP (1.10×10¹³ GC/mL) was produced by the Research Vector Core at the Center for Advanced Retinal and Ocular Therapeutics (University of Pennsylvania). The production method for these viruses have been previously described (Ramachandran et al. Hum. Gene Ther. 28, 154-167 (2017)). All viruses were produced using the same transgene construct, consisting of the CAG promoter derived from InvivoGen pDRIVE CAG plasmid (InvivoGen, San Diego, Calif., USA), the cDNA encoding enhanced GFP (eGFP) protein, and the bovine growth hormone polyadenylation signal.

### Animal Surgery

CBA/J mice were used in this study. For neonatal mice (P0-P5), hypothermia was used to induce and maintain anesthesia. Surgery was performed only in the left ear of each animal. The right ear served as a control. For inner ear gene delivery via the posterior semicircular canal approach, a post-auricular incision was made, and the tissue was dissected to expose the posterior semicircular canal. Care was taken to avoid the facial nerve during the dissection. A Nanoliter Microinjection System (Nanoliter2000; World Precision Instruments, Sarasota, Fla., USA) was used in conjunction with a glass micropipette to load AAV-eGFP into the glass micropipette. A total of 1 μL of AAV-eGFP was injected over approximately 40 s. The incision was closed with 5-0 vicryl sutures.

For adult mice, anesthesia was induced using isoflurane gas (Baxter, Deerfield, Ill., USA) through a nose cone at a flow rate of 0.5 L/min. The adult mouse otic capsule was completely ossified (in contrast to the neonatal mouse otic capsule, which is cartilaginous). Therefore, the adult mouse inner ear gene delivery was done using the round window approach. A post-auricular incision was made using small scissors. The soft tissues were bluntly dissected to expose the bulla. A small hole was created in the bulla with a 25-gauge needle and enlarged with forceps to expose the round window (RW) membrane. A Nanoliter Microinjection System (Nanoliter2000; World Precision Instruments, Sarasota, Fla., USA) was used in conjunction with a glass micropipette to load AAV-eGFP into the glass micropipette. A total of 2 μL of AAV2.7m8-eGFP (9.75×10¹² GC/mL) was injected over approximately 80 s. The incision was closed with 5-0 vicryl sutures.

### Auditory Brainstem Response

ABR testing was used to evaluate hearing sensitivity at ˜P30. Animals were anesthetized with ketamine (100 mg/kg) and xylazine (10 mg/kg) via intraperitoneal injections and placed on a warming pad inside a sound booth (ETS-Lindgren Acoustic Systems, Cedar Park, Tex., USA). The animal's temperature was maintained using a closed feedback loop and monitored using a rectal probe (CWE Incorporated, TC-1000, Ardmore, PN, USA). Sub-dermal needle electrodes were inserted at the vertex (+) and test-ear mastoid (−) with a ground electrode under the contralateral ear. Stimulus generation and ABR recordings were completed using Tucker Davis Technologies hardware (RZ6 Multi I/O Processor; Tucker-Davis Technologies, Gainesville, Fla., USA) and software (BioSigRx, v.5.1). ABR thresholds were measured at 4, 8, 16, and 32 kHz using 3-ms, Blackman-gated tone pips presented at 29.9/s with alternating stimulus polarity. At each stimulus level, 512-1024 responses were averaged. Thresholds were determined by visual inspection of the waveforms and were defined as the lowest stimulus level at which any wave could be reliably detected. A minimum of two waveforms were obtained at the threshold level to ensure repeatability of the response. Physiological results were analyzed for individual frequencies and then averaged for each of these frequencies from 4 to 32 kHz.

### Circling Behavior

The circling behavior of mice that underwent inner ear gene delivery was quantified using optical tracking and the ANY-maze tracking software (version 4.96; Stoelting Co., Wood Dale, Ill., USA). A 38×58 cm box was attached to a video camera (Fujinon YV5X2.7R4B-2⅓-inch 2.7-13.5 mm F1.3 Day/Night Aspherical Vari-focal Lens). The ANY-maze video tracking software was set to track the head of the mice placed within the box. Each mouse was placed into the box and allowed to acclimate to the new environment for 2 min. Complete rotations were recorded and quantified for the next 2 min, followed by a 1-minute “cool-down” period where rotations were not tracked. Each mouse was assessed three times, and the average was taken.

### Immunohistochemistry and Quantification

After completion of functional testing, mice were euthanized by CO₂ asphyxiation followed by decapitation. Temporal bones were harvested and fixed overnight with 4% paraformaldehyde followed by decalcification in 120 mM ethylenediaminetetraacetic acid for 4 days. The vestibular organs and cochlear sensory epithelia were micro-dissected, blocked, and labeled with mouse anti-myosin 7a antibody to label hair cells (1:200, product #25-6790; Proteus BioSciences, Ramona, Calif., USA), with mouse anti-acetylated tubulin antibody to label supporting cells (1:100, product #T9026; Sigma-Aldrich Corp., St. Louis, Mo., USA), and chicken anti-GFP antibody (1:1000, product #ab13970; Abcam, Cambridge, Mass., USA), and Hoechst stain (1:500, product #62249; Life Technologies, Carlsbad, Calif., USA) to label nuclei. Primary and secondary antibodies were diluted in phosphate-buffered saline. Images were obtained using a Zeiss LSM780 confocal microscope at 10× and 40× using z-stacks.

For hematoxylin and eosin staining, tissues were first treated with a sucrose gradient (10-30% in phosphate-buffered saline) and then were treated with a mixture of sucrose and embedding medium SCEM (Section-Lab Co Ltd, Japan). After freezing in liquid nitrogen, tissues were then sectioned at 10 μm thickness and hematoxylin and eosin staining was done using the Hematoxylin & Eosin Stain Kit following the manufacturer's instructions (Vector Laboratories, Inc., Burlingame, Calif., USA).

For the quantification of cochlear hair cell and supporting cell infection efficiency, two 40× images were taken at the apex, middle turn, and base of cochlea. The number of hair cells and supporting with GFP expression was counted and averaged at each location along the cochlea. Each 40× image contains ˜30 IHCs and ˜90 OHCs. The overall infection rate was calculated by averaging the infection rates obtained from the entire cochlea. For the quantification of utricular hair cell infection efficiency, two 40× images (each containing ˜300 vestibular hair cells) were taken per utricle specimen and the number of hair cells with GFP expression was counted and averaged.

### Statistics

Student's t test (two-tailed) was used to assess the differences in infection efficiency. It has been shown that different AAV serotypes can have different infection efficiencies in different regions of the cochlea. Therefore, infection efficiencies from each region of the cochlea (apex, middle turn, and cochlear base) were treated as separate measurements in the calculation of mean, standard error, and statistical significance. ANOVA was used to assess the differences in ABR thresholds as well as the circling behavior. Post-hoc analysis was performed using Scheffe's method. A p value <0.05 indicates statistical significance.

### Results

The infection patterns of AAV8BP2 in the mouse inner ear were examined. To assess the infection efficiency of AAV8BP2 in the mammalian inner ear, AAV8BP2-GFP (1.10×1013 GC/mL) was delivered to neonatal (P0-P5) mouse inner ears using the posterior semicircular canal approach. Posterior semicircular canal gene delivery allows viral vectors to effectively infect cells in the neonatal cochlea as well as vestibular organs. Infection efficiencies of AAV2-GFP (5.69×1012 GC/mL) and AAV8-GFP (1.66×1013 GC/mL), the two commonly used conventional AAVs from which AAV2.7m8 and AAV8BP2 are derived respectively, as well as the synthetic AAV Anc80L65-GFP (1.89×1013 GC/mL), were also examined using the same delivery approach as additional controls. One microliter of AAV was delivered into each animal. Hair cell infection efficiency was assessed by quantifying the percentage of hair cells (identified by anti-Myo7a antibody) with green fluorescent protein (GFP) expression. Mice injected with AAV8BP2-GFP (n=9 animals) had moderate-to-high levels of GFP expression in IHCs and OHCs. The overall infection efficiency was 55.7±9.53% for IHC and 44.1±7.94% for OHC.

In addition to assessing the hair cell infection efficiency of AAV8BP2 in the cochlea, the hair cell infection efficiency was also examined in the vestibular organs. When AAV8BP2-GFP was delivered to neonatal mouse inner ears, GFP was expressed in vestibular organs. Quantification of vestibular hair cell infection efficiency was done in the utricle. The utricular hair cell infection efficiency was 34.2±9.84% for AAV8BP2-GFP.

In order for the inner ear gene therapy to be a viable treatment for hearing loss and vestibular dysfunction, the viral vector used should have minimal effect on normal auditory and vestibular functions. To assess whether the inner ear delivery of AAV8BP2 had any effect on hearing, auditory brainstem responses (ABRs) were measured. In mice that underwent AAV8BP2-GFP injection at a concentration of 0.55×1010 GC, the ABR thresholds were comparable to those of control mice.

Mice with vestibular dysfunction often exhibit circling behavior. To assess whether the inner ear delivery of synthetic AAVs had any effect on the vestibular system, the circling behavior of injected mice was examined. Control mice that did not undergo inner ear gene delivery circled 5.11±0.32 times per 2 min (n=6 animals). Injection of AAV8BP2-GFP at a concentration of 0.55×1010 GC resulted in no increase in circling behavior compared to control animals (5.47±0.77 times per 2 min, p=0.66, n=5 animals). This result indicates that the inner ear delivery of AAV8BP2-GFP is safe and resulted in no adverse effect in auditory and vestibular functions.

What is claimed is:

1. A recombinant adeno-associated virus (AAV) virion comprising:
a) a modified AAV capsid protein, wherein the modified AAV capsid protein comprises a mutation relative to a corresponding parental AAV capsid protein, wherein the mutation in the modified AAV capsid protein is in amino acids corresponding to amino acids 587-595 of AAV8-VP1; and
b) a heterologous nucleic acid that produces an expression product, wherein the expression product reduces hearing loss or dizziness.

2. The recombinant AAV virion of claim 1, wherein the expression product is a nucleic acid that decreases expression of a gene associated with hearing loss, wherein the gene is selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C.

3. The recombinant AAV virion of claim 1, wherein the expression product is a polypeptide that reduces hearing loss, wherein the polypeptide is selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPL5, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A.

4. The recombinant AAV virion of claim 1, wherein the AAV virion is an AAV2 virion, an AAV5 virion, an AAV8 virion or an AAV9 virion.

5. The recombinant AAV virion of claim 1, wherein the AAV virion is an AAV8BP2 virion.

6. The recombinant AAV virion of claim 1, wherein the nucleic acid that decreases expression of a gene associated with hearing loss is an interfering RNA.

7. The recombinant AAV virion of claim 6, wherein the interfering RNA is an antisense molecule, a short interfering RNA or an miRNA.

8. The recombinant AAV virion of claim 1, wherein the hearing loss is selected from the group consisting of age-related hearing loss, hereditary hearing loss, noise-induced hearing loss, hearing loss as the result of administration of ototoxic drugs, disease-associated hearing loss and hearing loss resulting from trauma.

9. A method for treating or preventing cochlear damage in a subject comprising administering to the subject having cochlear damage or at risk of developing cochlear damage, an effective amount of the recombinant AAV virion of claim 1.

10. The method of claim 9, wherein the subject has or is at risk of developing age-related hearing loss, hereditary hearing loss, noise-induced hearing loss, hearing loss as the result of administration of ototoxic drugs, disease-associated hearing loss and hearing loss resulting from trauma.

11. The method of claim 9, wherein the recombinant AAV virion infects the cochlear lateral wall of the subject.

12. The method of claim 9, wherein the recombinant AAV virion infects the stria vascularis in the cochlear lateral wall.

13. The method of claim 12, wherein the recombinant AAV virion infects the marginal cells, the intermediate cells and/or the basal cells of the stria vascularis.

14. The method of claim 9, wherein the recombinant AAV virion increases inner ear hair cell regeneration.

15. The method of claim 14, wherein the recombinant AAV virion increases cochlear hair cell regeneration.

16. A method for treating or preventing hearing loss or dizziness in a subject, comprising administering to the subject having hearing loss or dizziness or at risk of developing hearing loss or dizziness, an effective amount of the recombinant AAV virion of claim 1.

17. The method of claim 16, wherein the subject has or is at risk of developing age-related hearing loss, hereditary hearing loss, noise-induced hearing loss, hearing loss as the result of administration of ototoxic drugs, disease-associated hearing loss and hearing loss resulting from trauma.

18. The method of claim 16, wherein the recombinant AAV virion infects the cochlear lateral wall of the subject.

19. The method of claim 18, wherein the recombinant AAV virion infects the stria vascularis in the cochlear lateral wall.

20. The method of claim 16, wherein the recombinant AAV virion infects the marginal cells, the intermediate cells and/or the basal cells.

21. The method of claim 16, wherein the recombinant AAV virion increases inner ear hair cell regeneration.

22. The method of claim 21, wherein the recombinant AAV virion increases cochlear hair cell regeneration.

23. The method of claim 9, wherein the recombinant AAV virion is administered intravenously, intrathecally, intratympanically, via round window administration, via semicircular canal delivery, or via stapedotomy.

24. The method of claim 23, wherein the recombinant AAV virion is administered via canalostomy into the posterior semicircular canal of the subject.
